(19) United States
(12) Patent Application Publication
(10) Pub. No.: US 2008/0318824 A1
(43) Pub. Date: Dec. 25, 2008

(54) Hairdressing Preparation Cleansers and Usage Method Thereof

(76) Inventors: Shigeru Iwai, Yokohama-shi (JP); Keiichi Uchikawa, Yokohama-shi (JP)

Correspondence Address: RANKIN, HILL & CLARK LLP, 925 EUCLID AVENUE, SUITE 700, CLEVELAND, OH, 44115-1405, US

(21) Appl. No.: 11/570,523

(22) PCT Filed: Jun. 14, 2005
(86) PCT No.: PCT/JP05/10883

§ 371 Date: Dec. 13, 2006

(30) Foreign Application Priority Data
Jun. 16, 2004 (JP) 2004-178145

(51) Int. Cl. A61K 8/72; A61K 8/73

(52) U.S. Cl. 510/119

(57) ABSTRACT

It is an object of the invention to provide a hair cleanser that has a high cleansing effect on hairdressing preparation and can provide smoothness to the hair.

A hairdressing preparation cleanser comprising:
(a) a component with the IOB value of 0.6 to 1.5;
(b) a cationic polymer; and
(c) a sequestering agent.

It is preferable that said component with the IOB value of 0.6 to 1.5 is one or a combination of more than one selected from the group consisting of random alkylene oxide derivatives, and said cationic polymer is one or a combination of more than one selected from the group of starch hydroxypropyltrimonium chloride, cationized hydroxyethyl cellulose-2, polyquaternium-39, and polyquaternium-7. It is preferable that citric acid and triethanolamine are additionally contained.

### RELATED APPLICATIONS

This application claims priority to the Japanese Patent Application 2004-178145 dated on Jun. 16, 2004 and is hereby incorporated with reference for all purposes.

### BACKGROUND OF THE INVENTION

1. Field of the Invention

The present invention relates to hairdressing preparation cleansers and a usage method thereof.

2. Prior Art

Hairdressing preparations are products that are used to style the hair and maintain the styled hair as well as to hydrate the hair. Examples of hairdressing preparations include hair foam, hair spray, hair gel, and setting lotion, in which hair styling is achieved with a resin film; and hair wax, hair stick, pomade, and hair cream, the main component of which is a solid oil or paste oil.

Under the condition of applied hairdressing preparation, shampoo has a poor foaming property, and users cannot feel the satisfaction that their hair has been washed. In addition, if hairdressing preparation is still left after the hair is washed, the hair after drying will result in a poor finish. Thus, when hairdressing preparation is applied, it is necessary to wash the hair multiple times.

Repeated shampooing of the hair, however, will unnecessarily degrease the scalp and hair and become the main cause of rough hands. In addition, it is unfavorable to individuals and hair salons on the cost front.

Thus, it is necessary to discover a cleanser, with which hairdressing preparation can be removed by a single washing, or a cleanser that can be used before shampooing of the hair. So far there has been no such a cleanser.

The present invention was made in view of the above-described problem, and the objects of the invention is to provide hair cleansers that have a high cleansing effect on hairdressing preparation and can provide smoothness to the hair as well as to provide a usage method of the cleansers.

### SUMMARY OF THE INVENTION

The present inventors have diligently studied to achieve the above-mentioned objectives. As a result, the inventors have found that a hair cleanser that has a high cleansing effect on hairdressing preparation and can provide smoothness to the hair can be obtained by blending a component with a specific IOB value, a cationic polymer, and a sequestering agent, thus leading to completion of the present invention.

Specifically, this invention is a hairdressing preparation cleanser comprising:

(a) a component with the IOB value of 0.6 to 1.5;

(b) a cationic polymer; and

(c) a sequestering agent.

It is preferable that said component is one or a combination of more than one selected from the group consisting of random alkylene oxide derivatives represented by the following formula (I) and diethoxyethyl succinate:

R¹O—[(AO)ₘ(EO)ₙ]—R² (I)

wherein AO is an oxyalkylene group having 3 to 4 carbon atoms, EO is an oxyethylene group, m is the average mole number of the added oxyalkylene groups and 1≦m≦70, n is the average mole number of the added oxyethylene groups and 1≦n≦70, the percentage of the oxyethylene groups with respect to the sum of the oxyalkylene groups having 3 to 4 carbon atoms and the oxyethylene groups is 20 to 80 weight %, and the oxyalkylene groups having 3 to 4 carbon atoms and the oxyethylene groups are randomly added; and each of R1 and R2 is a hydrocarbon group having 1 to 4 carbon atoms or a hydrogen atom and they can be either the same or different, and the ratio of the number of hydrogen atoms to the number of hydrocarbon groups R¹ and R² is equal to or less than 0.15.

It is preferable that said cationic polymer is one or a combination of more than one selected from the group of starch hydroxypropyltrimonium chloride, cationized hydroxyethyl cellulose-2, polyquaternium-39, and polyquaternium-7.

It is preferable that said sequestering agent is one or a combination of more than one selected from the group of hydroxyethane diphosphonic acid, its salts, edetates, and N-acyl ethylenediaminetriacetates.

In the hairdressing preparation cleanser of the present invention, it is preferable that citric acid and triethanolamine are additionally contained.

And, usage method for the hairdressing preparation cleanser of the present invention comprises said hairdressing preparation cleanser is applied on the dry hair bearing hairdressing preparation, and the hair is subsequently rinsed with water.

According to the present invention, it is possible to obtain a hair cleanser that has a high cleansing effect on hairdressing preparation and can provide smoothness to the hair by blending a component with a specific IOB value, a cationic polymer, and a sequestering agent.

### DESCRIPTION OF THE PREFERRED EMBODIMENT

A preferable mode for carrying out the present invention is described in the following.

(a) A Component with the IOB Value of 0.6 to 1.5

As the component with the IOB value of 0.6 to 1.5, one or a combination of more than one selected from the group consisting of random alkylene oxide derivatives and diethoxyethyl succinate is desirable. If the IOB value is less than 0.6, the cleansing effect will be inferior and the feeling of the finished hair will be poor.

The blending quantity of the component with the IOB value of 0.6 to 1.5 is 0.5 to 20 weight % and preferably 1 to 20 weight %. If the blending quantity is less than 0.5 weight %, the effect of the present invention is not satisfactory. If the blending quantity is equal to or more than 20 weight %, the viscosity of the liquid is too high and it is difficult to use it.

The random alkylene oxide derivatives are represented by the following formula (I):

R¹O—[(AO)ₘ/(EO)ₙ]—R² (I)

wherein AO is an oxyalkylene group having 3 to 4 carbon atoms, EO is an oxyethylene group, m is the average mole number of the added oxyalkylene groups and 1≦m≦70, n is the average mole number of the added oxyethylene groups and 1≦n≦70, the percentage of the oxyethylene groups with respect to the sum of the oxyalkylene groups having 3 to 4 carbon atoms and the oxyethylene groups is 20 to 80 weight %, and the oxyalkylene groups having 3 to 4 carbon atoms and the oxyethylene groups are randomly added; and each of R1 and R2 is a hydrocarbon group having 1 to 4 carbon atoms or a hydrogen atom and they can be either the same or different, and the ratio of the number of hydrogen atoms to the number of hydrocarbon groups R¹ and R² is equal to or less than 0.15.

In the above described formula (I), AO is an oxyalkylene group having 3 to 4 carbon atoms. Specific examples include an oxypropylene group, an oxybutylene group, an oxyisobutylene group, an oxytrimethylene group, and an oxytetramethylene group. Preferably AO is an oxypropylene group or an oxybutylene group.

The average mole number of the added oxyalkylene groups having 3 to 4 carbon atoms is expressed with m, and 1≦m≦70, preferably 2≦m≦41. The average mole number of the added oxyethylene groups is expressed with n, and 1≦n≦70, preferably 14≦n≦55. If the number of the oxyalkylene groups having 3 to 4 carbon atoms or the number of the oxyethylene groups is zero, the smoothness decreases. If the number of the oxyalkylene groups having 3 to 4 carbon atoms or the number of the oxyethylene groups is more than 70, a sticky feeling tends to be generated.

In addition, the percentage of the oxyethylene groups with respect to the sum of the oxyalkylene groups having 3 to 4 carbon atoms and the oxyethylene groups is preferably 20 to 80 weight %. If the percentage of the oxyethylene group is less than 20 weight %, the smoothness tends to decrease. If the percentage of the oxyethylene groups is more than 80 weight %, the stickiness after use tends to be caused.

The oxyethylene groups and the oxyalkylene groups having 3 to 4 carbon atoms are randomly added, and the order of addition need not be specified. The product with a random polymer generates better texture during use than the product with a block polymer.

In the present invention, EO is an oxyethylene group, PO is an oxypropylene group, and [(EO)/(PO)] represents random bonding.

Each of R1 and R2 is a hydrocarbon group having 1 to 4 carbon atoms or a hydrogen atom. Examples of hydrocarbon groups include a methyl group, an ethyl group, an n-propyl group, an isopropyl group, an n-butyl group, a sec-butyl group, and a tert-butyl group. Preferably they are a methyl group or an ethyl group. In the case of a hydrocarbon group having more than five carbon atoms, the moist feeling is lowered because of decreased hydrophilicity. R¹ and R² may be identical or different.

For R¹ and R², the same group may be used, respectively, a hydrocarbon group having 1 to 4 carbon atoms and a hydrogen atom may be mixed, or different hydrocarbon groups having 1 to 4 carbon atoms may be mixed. However, the ratio (Y/X) of the number of hydrogen atoms (Y) to the number of hydrocarbon groups R¹ and R² (X) is preferably equal to or less than 0.15, more preferably it is equal to or less than 0.06. If the ratio Y/X is more than 0.15, a sticky feeling is caused.

Specific examples of alkylene oxide derivatives of the present invention include POE (14) POP (7) dimethyl ether (IOB: 1.19), POE (36) POP (41) dimethyl ether (IOB: 0.90), POE (55) POP (28) dimethyl ether (IOB: 1.20), and POE (36) POP (41) dimethyl ether (IOB: 0.90).

In the present invention, the above-described POE, POP, and POB are abbreviations for polyoxyethylene, polyoxypropylene, and polyoxybutylene, respectively.

Alkylene oxide derivatives of the present invention can be prepared according to publicly known methods. For example, they can be obtained by the addition polymerization of ethylene oxide and alkylene oxide containing 3 to 4 carbon atoms with a compound containing hydroxyl groups and the subsequent ether reaction with halogenated alkyl compound in the presence of an alkaline catalyst. Synthesis examples of alkylene oxide derivatives are shown below.

### SYNTHESIS EXAMPLE

### Random Alkylene Oxide Derivative

Polyoxyethylene (10 moles) polyoxypropylene (10 moles) dimethyl ether

CH₃O[(EO)₁₀/(PO)₁₀]CH₃ (random polymer)

Into an autoclave, 76 g of propylene glycol and 3.1 g of potassium hydroxide, which is a catalyst, were loaded. After the air in the autoclave was replaced with dry nitrogen, the catalyst was completely dissolved by stirring at 140° C. Subsequently, a mixture of 440 g ethylene oxide and 522 g of propylene oxide was dropwise added with a liquid dropping apparatus, and the mixture was stirred for 2 hours. Then, 224 g of potassium hydroxide was loaded, the inside of the system was replaced with dry nitrogen, 188 g of methyl chloride was pressured in at 80 to 130° C., and the reaction was carried out for 5 hours. After that, the reaction product was taken out from the autoclave and neutralized with hydrochloric acid to pH 6 to 7. In order to remove contained moisture, the product was treated under a reduced pressure of −0.095 MPa (50 mmHg) for 1 hour at 100° C. In addition, filtration was conducted to remove the salt formed by the treatment, and the above-described random alkylene oxide derivative was obtained.

### REFERENCE SYNTHESIS EXAMPLE

### Block Alkylene Oxide Derivative

Polyoxyethylene (10 moles) polyoxypropylene (10 moles) dimethyl ether

CH₃O[(EO)₁₀(PO)₁₀]CH₃ (block polymer)

Into an autoclave, 76 g of propylene glycol and 3.1 g of potassium hydroxide, which is a catalyst, were loaded. After the air in the autoclave was replaced with dry nitrogen, the catalyst was completely dissolved by stirring at 140° C. Then, 522 g of propylene oxide was dropwise added with a liquid dropping apparatus, and the mixture was stirred for 2 hours. Subsequently, 440 g of ethylene oxide was dropwise added with a liquid dropping apparatus, and the mixture was stirred for 2 hours. Then, 224 g of potassium hydroxide was loaded, the inside of the system was replaced with dry nitrogen, 188 g of methyl chloride was pressured in at 80 to 130° C., and the reaction was carried out for 5 hours. After that, the reaction product was taken out from the autoclave and neutralized with hydrochloric acid to pH 6 to 7. In order to remove contained moisture, the product was treated under a reduced pressure of −0.095 MPa (50 mmHg) for 1 hour at 100° C. In addition, filtration was conducted to remove the salt formed by the treatment, and the above-described block alkylene oxide derivative was obtained.

The hydroxyl value of the compound that was obtained by purifying a sample taken before the methyl chloride reaction was 110, and the hydroxyl value of the obtained compound was 0.3. The ratio of the number of terminal hydrogen atoms to the number of the terminal methyl groups was 0.003; thus, the hydrogen atoms are almost completely replaced with methyl groups.

### (b) Cationic Polymer

Specific examples of the cationic polymers include starch hydroxypropyltrimonium chloride, cationized hydroxyethyl cellulose-2, polyquaternium-39, and polyquaternium-7, and one or a combination of more than one can be used.

The blending quantity of the cationic polymer is preferably 0.1 to 1.0 weight %. If the blending quantity is less than 0.1 weight %, the cleansing effect is low. If the blending quantity is equal to or more than 1.0 weight %, the viscosity of the liquid is too high and it is difficult to use it.

### (c) Sequestering Agent

As a sequestering agent, it is desirable to use hydroxyethane diphosphonic acid and its salts such as 1-hydroxyethane-1,1-diphosphonic acid and tetrasodium 1-hydroxyethane-1,1-diphosphonate; edetic acid and its salts such as disodium edetate, trisodium edetate, and tetrasodium edetate; and N-acyl ethylenediaminetriacetates such as lauroyl ethylenediaminetriacetate and trisodium ethylenediaminehydroxyethyltriacetate, and one or a combination of more than one can be used.

The blending quantity of the sequestering agent is preferably 0.1 to 3.0 weight %. If the blending quantity is less than 0.1 weight %, the viscosity of the liquid is too low and it is difficult to use it, and the feeling of the finished hair may be inferior. If the blending quantity is equal to or more than 3.0 weight %, the feeling of the finished hair may also be inferior.

In addition, it is possible to use in combination with sodium polyphosphate, sodium metaphosphate, gluconic acid, phosphoric acid, citric acid, sodium citrate, ascorbic acid, succinic acid, or ethylenediaminetetraacetic acid. In particular, a combination with citric acid and triethanolamine is desirable because it is possible to keep the product stable by maintaining the pH at approximately 6 to 8.

In the hairdressing preparation cleanser of the present invention, ingredients used in regular cosmetics and ingredients used in quasi-drug hair care cosmetics can be blended in addition to the above-described essential components, and they can be produced with the usual method.

The hairdressing preparation cleanser of the present invention is applied, before shampooing, on the dry hair still bearing hairdressing preparation. Subsequently, the hair is rinsed with water. The cleanser can be used on the wet hair; however, it is preferable to use it on the dry hair to avoid the trouble of pre-wetting the hair and also to achieve effective cleansing.

The cleanser can be used as a spray or mist by filling the cleanser into the container of a trigger dispenser and spraying a suitable amount on the hair. In addition, when N-acyl ethylenediaminetriacetate is used as a sequestering agent, the cleanser can be used by preparing foam in the container of a pump foamer.

After the use of the hairdressing preparation cleanser, it is preferable to wash the hair with normal shampoo. Because hairdressing preparation is removed by the present cleanser, multiple shampooing is not necessary, and the foaming property of shampoo is improved.

It is desirable to additionally use hair rinse, hair treatment, hair conditioner, etc. after shampooing. By the use of the present hairdressing preparation cleanser, the feeling of the finished hair can be improved.

In the following, the present invention will be more concretely described with reference to examples. However, the present invention is not limited by these examples. The blending quantity will be expressed in weight % unless otherwise noted.

### EXAMPLE 1

At the beginning, evaluation test methods and evaluation criteria used in the examples are described.

### [Procedure]

1. Uniformly apply hairdressing preparation (GERAID Fiber in Wax N™, manufactured by Shiseido Co., Ltd.) on the hair.

2. Spray a test sample on the hair while the hair is still dry (3 g/10 cm²).

3. Lightly massage the hair with fingers and wash off under the shower.

4. Wash the hair with a commercial shampoo.

5. Apply a commercial hair conditioner and then rinse off.

The cleanser was evaluated, at 0.5 point intervals, by professional panelists (n=10) with hair coloring and perm, and the average was calculated. In each category, the average equal to or more than 1 point was regarded as passing and the average less than 1 point was regarded as failing.

### Evaluation (1): Ease of Use

Evaluate the ease of use of hairdressing preparation cleanser at step 2.

Uniform spreading and no dripping: 2 points

No spreading or too much spreading and dripping: 0 points

### Evaluation (2): Cleansing Effect and Hair Smoothness

Evaluate the cleansing effect on hairdressing preparation and the hair smoothness at step 3.

Cleansed well and smooth: 2 points

Feeling of some residue, slightly coarse and stiff: 1 point

Not cleansed, coarse and stiff: 0 points

### Evaluation (3): Foaming Property of Shampoo

Evaluate the foaming property of shampoo at step 4.

Good foaming: 2 points

Slightly poor foaming: 1 point

Poor foaming: 0 points

### Evaluation (4): The Feeling of the Finished Hair

Evaluate the feeling of the finished hair at step 5.

Smooth and supple: 2 points

Coarse and stiff: 0 points

### Evaluation (5): Product Stability

Change of the appearance with time (one month storage after the production and at room temperature)

A: Transparent

B: Hazy

C: Slightly separated

D: Separated to two layers with precipitation

Evaluation tests (1) to (5) were conducted for test samples with the blending compositions listed in Table 1. The results are shown in Table 1.

TABLE 1

| | Test Example | | | | | | | | |
|---|---|---|---|---|---|---|---|---|---|
| | 1-1 | 1-2 | 1-3 | 1-4 | 1-5 | 1-6 | 1-7 | 1-8 | 1-9 |
| CH₃O[(EO)₅₅/(PO)₂₈]CH₃ (IOB: 1.20) | 5 | 5 | 5 | 5 | 10 | — | — | — | — |
| CH₃O[(EO)₁₄/(PO)₇]CH₃ (IOB: 1.19) | — | — | — | — | — | 5 | 5 | — | — |
| CH₃O[(EO)₉/(PO)₂]CH₃ (IOB: 1.41) | — | — | — | — | — | — | — | 5 | — |
| CH₃O[(EO)₃₆/(PO)₄]CH₃ (IOB: 0.90) | — | — | — | — | — | — | — | — | 5 |
| diethoxyethyl succinate (IOB: 0.67) | — | — | — | — | — | — | — | — | — |
| cationized hydroxyethyl cellulose-2 | 0.8 | 0.3 | — | — | — | 0.5 | 0.1 | 0.3 | 0.3 |
| starch hydroxypropyltrimonium chloride | — | — | — | — | 0.3 | — | — | — | — |
| polyquaternium-39 | — | — | 0.3 | — | — | — | — | — | — |
| polyquaternium-7 | — | — | — | 0.3 | — | — | — | — | — |
| hydroxyethyl cellulose | — | — | — | — | — | — | — | — | — |
| xanthan gum | — | — | — | — | — | — | — | — | — |
| lauroyl ethylenediaminetriacetate | 0.5 | — | 0.3 | 0.3 | 0.3 | 0.3 | 0.1 | 0.3 | 0.3 |
| edetate | — | 0.5 | — | — | — | — | — | — | — |
| citric acid | 0.1 | 0.1 | 0.1 | — | — | — | — | — | — |
| triethanolamine | 0.3 | 0.2 | 0.3 | — | — | — | — | — | — |
| sodium citrate | — | — | — | 0.2 | 0.2 | 0.2 | 0.1 | 0.2 | 0.2 |
| dipropylene glicol | 5 | 5 | 5 | — | — | — | — | — | 5 |
| sorbitol | — | — | — | 5 | — | 5 | — | — | — |
| propylene glycol | — | — | — | — | — | — | — | 5 | — |
| 1,3-butylene glycol | — | — | — | — | — | — | — | — | — |
| glycerine | — | — | — | — | 5 | — | — | — | — |
| purified water | balance | balance | balance | balance | balance | balance | balance | balance | balance |
| Ease of use | 2.0 | 1.0 | 1.0 | 1.6 | 1.3 | 1.9 | 1.6 | 1.7 | 1.8 |
| Cleansing effect and hair smoothness | 1.9 | 1.6 | 1.6 | 1.7 | 1.9 | 1.8 | 1.4 | 1.7 | 1.0 |
| Foaming property of shampoo | 2.0 | 1.7 | 1.8 | 1.9 | 2.0 | 1.7 | 1.2 | 1.9 | 1.2 |
| The feeling of the finished hair | 2.0 | 1.3 | 1.9 | 1.6 | 1.8 | 1.6 | 1.4 | 1.3 | 1.0 |
| Product stability | A | A | A | B | B | B | B | B | B |

Test Example

| | 1-10 | 1-11 | 1-12 | 1-13 | 1-14 | 1-15 | 1-16 | 1-17 |
|---|---|---|---|---|---|---|---|---|
| CH₃O[(EO)₅₅/(PO)₂₈]CH₃ (IOB: 1.20) | — | 5 | 5 | — | — | — | — | — |
| CH₃O[(EO)₁₄/(PO)₇]CH₃ (IOB: 1.19) | — | — | — | — | — | — | — | — |
| CH₃O[(EO)₉/(PO)₂]CH₃ (IOB: 1.41) | — | — | — | — | — | — | — | — |
| CH₃O[(EO)₃₆/(PO)₄₁]CH₃ (IOB: 0.90) | — | — | — | — | — | — | — | — |
| diethoxyethyl succinate (IOB: 0.67) | 5 | — | — | — | — | — | — | — |
| cationized hydroxyethyl cellulose-2 | 0.3 | — | 0.3 | 0.3 | — | — | — | — |
| starch hydroxypropyltrimonium chloride | — | — | — | — | 0.3 | 0.3 | — | — |
| polyquaternium-39 | — | — | — | — | — | — | — | — |
| polyquaternium-7 | — | — | — | — | — | — | 0.3 | 0.3 |
| hydroxyethyl cellulose | — | — | — | — | — | — | — | — |
| xanthan gum | — | — | — | — | — | — | — | — |
| lauroyl ethylenediaminetriacetate | 0.3 | 0.3 | — | 0.5 | 0.3 | 0.3 | 0.3 | 0.3 |
| edetate | — | — | — | — | — | — | — | — |
| citric acid | — | — | 0.1 | — | — | — | — | — |
| triethanolamine | — | — | 0.2 | — | — | — | — | — |
| sodium citrate | 0.2 | 0.2 | — | 0.1 | 0.2 | 0.1 | 0.2 | 0.2 |
| dipropylene glicol | — | 5 | 5 | — | 5 | — | 5 | 10 |
| sorbitol | — | — | — | 7 | — | — | — | — |
| propylene glycol | — | — | — | — | — | 10 | — | — |
| 1,3-butylene glycol | 5 | — | — | — | — | — | 5 | — |
| glycerine | — | — | — | — | 5 | — | — | — |
| purified water | balance | balance | balance | balance | balance | balance | balance | balance |
| Ease of use | 1.5 | 0.9 | 0.9 | 1.1 | 1.1 | 0.9 | 1.6 | 1.2 |
| Cleansing effect and hair smoothness | 1.8 | 0.3 | 1.5 | 0.9 | 0.3 | 0.4 | 0.9 | 0.6 |
| Foaming property of shampoo | 1.9 | 1.2 | 1.8 | 1.4 | 0.8 | 0.7 | 1.6 | 1.6 |
| The feeling of the finished hair | 1.7 | 1.2 | 0.9 | 0.9 | 1.6 | 0.2 | 0.4 | 0.7 |
| Product stability | B | D | A | B | B | B | B | B |

### (Production Method)

Cationized hydroxyethyl cellulose-2 in purified water was heated and the cellulose was dissolved. Subsequently, other components were added and dissolved, and the solution was confirmed to be transparent.

A cleansing effect and hair smoothness could not be achieved in Test Examples 1-13 to 1-17 in which alkylene oxide derivatives or diethoxyethyl succinate was not blended.

The cleansing effect and the product stability were rather inferior in Test Example 1-11 in which cationized hydroxyethyl cellulose-2, starch hydroxypropyltrimonium chloride, polyquaternium-39, or polyquaternium-7, which is a cationic polymer, was not blended.

The ease of use and the feeling of the finished hair were rather inferior in Test Example 1-12 in which lauroyl ethylenediaminetriacetate or edetate, which is a sequestering agent of the present invention, was not contained.

In contrast, the hairdressing preparation cleansers of the present invention, Test Examples 1-1 to 1-10, were excellent in all evaluation categories. Especially the product stability of Test Examples 1-1 to 1-3, in which citric acid and triethanolamine were contained, was further improved.

As described above, it was clarified that a hair cleanser that has a high cleansing effect on hairdressing preparation and can provide smoothness to the hair can be obtained by blending (a) a component with the IOB value of 0.6 to 1.5, (b) a cationic polymer, and (c) a sequestering agent.

In the sections below, the preferable blending quantities of (a) a component with the IOB value of 0.6 to 1.5 and (b) a cationic polymer were discussed. Evaluation tests (1) to (5) were conducted for the test samples with blending compositions listed in Table 2. The results are shown in Table 2.

TABLE 2

| | Test Example | | | | | | | |
|---|---|---|---|---|---|---|---|---|
| | 2-1 | 2-2 | 2-3 | 2-4 | 2-5 | 2-6 | 2-7 | 2-8 |
| CH₃O[(EO)₅₅/(PO)₂₈]CH₃ | 0.5 | 1 | 20 | 25 | 5 | 5 | 5 | 5 |
| dipropylene glycol | 5 | 5 | 5 | 5 | 5 | 5 | 5 | 5 |
| cationized hydroxyethyl cellulose-2 | 0.3 | 0.3 | 0.3 | 0.3 | — | 0.1 | 1 | 2 |
| citric acid | 0.1 | 0.1 | — | — | — | — | — | — |
| triethanolamine | 0.2 | 0.2 | — | — | — | — | — | — |
| sodium citrate | — | — | 0.2 | 0.2 | 0.2 | 0.2 | 0.2 | 0.2 |
| lauroly ethylenediaminetriacetate | 0.3 | 0.3 | 0.3 | 0.3 | 0.3 | 0.3 | 0.3 | 0.3 |
| purified water | balance | balance | balance | balance | balance | balance | balance | balance |
| Ease of use | 1.4 | 1.6 | 1.2 | 0.9 | 0.9 | 1.6 | 1.2 | 0.4 |
| Cleansing effect and hair smoothness | 1.0 | 1.2 | 1.8 | 1.8 | 0.3 | 1.1 | 1.9 | 2.0 |
| Foaming property of shampoo | 1.1 | 1.4 | 2.0 | 2.0 | 1.2 | 1.6 | 2.0 | 1.9 |
| The feeling of the finished hair | 1.0 | 1.2 | 1.9 | 1.9 | 1.2 | 1.6 | 1.9 | 1.8 |
| Product stability | A | A | 8 | B | D | B | B | B |

### (Production Method)

Cationized hydroxyethyl cellulose-2 in purified water was heated and the cellulose was dissolved. Subsequently, other components were added and dissolved, and the solution was confirmed to be transparent.

When a component with the IOB value of 0.6 to 1.5 was blended at 0.5 to 20 weight %, more specifically at 1 to 20 weight %, the cleansers were excellent in all evaluation categories (1) to (4). When the blending quantity was more than 20 weight %, the viscosity of the test sample became high, and it was difficult to achieve uniform spreading.

When 0.1 to 1 weight % of a cationic polymer was blended, the cleansers were excellent in all evaluation categories (1) to (5). When the blending quantity was more than 1 weight %, the viscosity of the test sample became high and it was difficult to achieve uniform spreading.

From the above results, it was confirmed that the blending quantity of (a) a component with the IOB value of 0.6 to 1.5 was preferably 0.5 to 20 weight %, more preferably 1 to 20 weight % and that the blending quantity of (b) a cationic polymer was preferably 0.1 to 1 weight %.

In addition, the present inventors found that the preferable blending quantity of (c) a sequestering agent was 0.1 to 3.0 weight %.

Hereinafter, preferable formulation examples for hairdressing preparation cleanser of the present invention will be described; however the present invention is not limited by these examples. All hairdressing preparation cleansers in the following examples had a high cleansing effect on hairdressing preparation and could provide smoothness to the hair.

### EXAMPLE 2

Pump foamer-type hairdressing preparation cleanser

| | (Weight |
|---|---|
| | %) |
| (1) Dipropylene glycol | 6.0 |
| (2) CH₃O[(EO)₅₅/(PO)₂₈]CH₃ | 6.0 |
| (3) Cationized hydroxyethyl cellulose-2 | 0.3 |
| (4) Sodium citrate | 0.2 |
| (5) Methyl paraben | 0.2 |
| (6) Phenoxyethanol | 0.5 |
| (7) Sodium lauroyl ethylenediaminetriacetate solution (30%) | 1.0 |
| (8) Perfume | 0.1 |
| (9) Purified water | 85.7 |

### (Production Method)

Component 3 was dissolved in component 9. To this solution was added component 5, which has been dissolved in component 1. The mixture was heated to 70° C. and solubilized, and the solution was confirmed to be transparent.

### EXAMPLE 3

Spray-type hairdressing preparation cleanser

| | (Weight |
|---|---|
| | %) |
| (1) Sorbitol | 7.0 |
| (2) Diethoxyethyl succinate | 3.0 |
| (3) Starch hydroxypropyltrimonium chloride solution (24%) | 2.0 |
| (4) Sodium citrate | 0.1 |
| (5) Sodium benzoate | 0.1 |
| (6) Methyl paraben | 0.1 |
| (7) Phenoxyethanol | 0.3 |
| (8) Hydroxyethane diphosphonic acid | 0.3 |
| (9) Perfume | 0.1 |
| (10) Purified water | 87.0 |

### (Production Method)

All components for blending were mixed at 70° C. and solubilized. After the solution was confirmed to be transparent, the solution was cooled.

### EXAMPLE 4

Pearlescent shampoo

| | (Weight |
|---|---|
| | %) |
| (1) CH₃O[(EO)₁₄/(PO)₇]CH₃ | 2.0 |
| (2) Sodium cocoyl methyltaurate solution (30%) | 16.0 |
| (3) Coco amidopropyl betaine solution (30%) | 14.0 |
| (4) Sodium lauroyl glycol acetate solution (30%) | 5.0 |
| (5) Sodium lauroyl ethylenediaminetriacetate solution (30%) | 1.2 |
| (6) Coco monoethanolamide | 0.3 |
| (7) Glycol distearate | 2.0 |
| (8) Cationized hydroxyethyl cellulose-2 | 0.3 |
| (9) Marcoat 550 ™ (8% aqueous solution of polyquaternium-7) | 3.0 |
| (10) Starch hydroxypropyltrimonium chloride solution (24%) | 1.5 |
| (11) Citric acid | 0.2 |
| (12) Dimethicone | 0.6 |
| (13) Sodium benzoate | 0.1 |
| (14) Phenoxyethanol | 0.3 |
| (15) Perfume | 0.4 |
| (16) Purified water | 50.0 |

### (Production Method)

This was prepared by the conventional method.

### EXAMPLE 5

Transparent shampoo

| | (Weight |
|---|---|
| | %) |
| (1) 1,3-Butylene glycol | 6.0 |
| (2) CH₃O[(EO)₅₅/(PO)₂₈]CH₃ | 10.0 |
| (3) Sodium polyoxyethylene lauryl ether sulfate solution (30%) | 25.0 |
| (4) Coco amidopropyl betaine solution | 15.0 |
| (5) Coco monoethanolamide | 0.5 |
| (6) Cationized hydroxyethyl cellulose-2 | 0.1 |
| (7) Starch hydroxypropyltrimonium chloride solution (24%) | 2.0 |
| (8) Sodium lauryl glycol acetate solution (30%) | 0.3 |
| (9) Citric acid | 0.2 |
| (10) Sodium benzoate | 0.1 |
| (11) Phenoxyethanol | 0.3 |
| (12) Perfume | 0.5 |
| (13) Purified water | 40.0 |

### (Production Method)

This was prepared by the conventional method.

1. A hairdressing preparation cleanser comprising:
(a) a component with the IOB value of 0.6 to 1.5;
(b) a cationic polymer; and
(c) a sequestering agent.

2. The hairdressing preparation cleanser of claim 1, wherein the component with the IOB value of 0.6 to 1.5 is one or a combination of more than one selected from the group consisting of random alkylene oxide derivatives represented by the following formula (I) and diethoxyethyl succinate:
R¹O—[(AO)ₘ/(EO)ₙ]—R² (I)
wherein AO is an oxyalkylene group having 3 to 4 carbon atoms, EO is an oxyethylene group, m is the average mole number of the added oxyalkylene groups and 1≦m≦70, n is the average mole number of the added oxyethylene groups and 1≦n≦70, the percentage of the oxyethylene groups with respect to the sum of the oxyalkylene groups having 3 to 4 carbon atoms and the oxyethylene groups is 20 to 80 weight %, and the oxyalkylene groups having 3 to 4 carbon atoms and the oxyethylene groups are randomly added; and each of R¹ and R² is a hydrocarbon group having 1 to 4 carbon atoms or a hydrogen atom and they can be either the same or different, and the ratio of the number of hydrogen atoms to the number of hydrocarbon groups R¹ and R² is equal to or less than 0.15.

3. The hairdressing preparation cleanser of claim 1, wherein the cationic polymer is one or a combination of more than one selected from the group of starch hydroxypropyltrimonium chloride, cationized hydroxyethyl cellulose-2, polyquaternium-39, and polyquaternium-7.

4. The hairdressing preparation cleanser of claim 1, wherein the sequestering agent is one or a combination of more than one selected from the group of hydroxyethane diphosphonic acid, its salts, edetates, and N-acyl ethylenediaminetriacetates.

5. The hairdressing preparation cleanser of claim 1, wherein citric acid and triethanolamine are additionally contained.

6. A method of use of hairdressing preparation cleanser, wherein the hairdressing preparation cleanser of claim 1 is applied on the dry hair bearing hairdressing preparation, and the hair is subsequently rinsed with water.
